(19) United States
(12) Patent Application Publication
(10) Pub. No.: US 2016/0335398 A1
(43) Pub. Date: Nov. 17, 2016

(54) MONITORING IMPACTS BETWEEN INDIVIDUALS FOR CONCUSSION ANALYSIS

(71) Applicant: INTERNATIONAL BUSINESS MACHINES CORPORATION, ARMONK, NY (US)

(72) Inventors: JAMES R. KOZLOSKI, NEW FAIRFIELD, CT (US); MARK C. H. LAMOREY, WILLISTON, VT (US); CLIFFORD A. PICKOVER, YORKTOWN HEIGHTS, NY (US); JOHN J. RICE, MOHEGAN LAKE, NY (US)

(21) Appl. No.: 14/745,497

(22) Filed: Jun. 22, 2015

Related U.S. Application Data
(63) Continuation of application No. 14/709,564, filed on May 12, 2015.

(51) Int. Cl. G06F 19/00; H04Q 9/00; A61B 5/00

(52) U.S. Cl. CPC G06F 19/322; A61B 5/4076; A61B 5/7275; A61B 5/6804; H04Q 9/00; H04Q 2209/40

(57) ABSTRACT

Embodiments include methods, systems and computer program products for monitoring impacts between users of uniforms for concussion analysis. Aspects include monitoring one or more sensors in a uniform of a user, determining whether the user experienced an impact and storing data from the one or more sensor associated with the impact in a memory. Aspects also include transmitting a user identification code associated with the uniform and storing a second user identification code that is associated with another uniform involved in the impact in the memory.

### DOMESTIC PRIORITY

This application is a continuation of U.S. application Ser. No. 14/709,564; Attorney Docket: YOR920150168US1; Filed: May 12, 2015; which is related to U.S. application Ser. No. 14/709,575; Attorney Docket No. YOR920150161US1; Filed: May 12, 2015; U.S. application Ser. No. 14/709,574; Attorney Docket: YOR920150162US1; Filed: May 12, 2015; U.S. application Ser. No. 14/709,572; Attorney Docket: YOR920150163US1; Filed: May 12, 2015; U.S. application Ser. No. 14/709,570; Attorney Docket: YOR920150164US1; Filed: May 12, 2015; U.S. application Ser. No. 14/709,563; Attorney Docket: YOR920150165US1; Filed: May 12, 2015; U.S. application Ser. No. 14/709,568; Attorney Docket: YOR920150167US1; Filed: May 12, 2015; U.S. application Ser. No. 14/664,987; Filed Mar. 23, 2015; Attorney Docket No.: YOR920150038US1; U.S. application Ser. No. 14/664,989; Filed: Mar. 23, 2015; Attorney Docket No.: YOR920150039US1; and U.S. application Ser. No. 14/664,991; Filed: Mar. 23, 2015; Attorney Docket No.: YOR920150040US1, the contents of each of which are herein incorporated by reference in their entirety.

### BACKGROUND

The present disclosure relates to monitoring the impacts between individuals, and more specifically, to methods, systems and computer program products for using sensors in a uniform to monitor impacts between individuals for concussion analysis.

Generally speaking, safety is a primary concern for both users of helmets and manufacturers of helmets. Helmets are used by individuals that participate in activities that have risk of head trauma, such as the area of sports, biking, motorcycling, etc. While helmets have traditionally been used to provide protection from blunt force trauma to the head, an increased awareness of concussion causing forces has motivated a need for advances in helmet technology to provide increased protection against concussions. A concussion is a type of traumatic brain injury that is caused by a blow to the head that shakes the brain inside the skull due to linear or rotational accelerations. Recently, research has linked concussions to a range of health problems, from depression to Alzheimer's, along with a range of brain injuries. Unlike severe traumatic brain injuries, which result in lesions or bleeding inside the brain and are detectable using standard medical imaging, a concussion is often invisible in brain tissue, and therefore only detectable by means of a cognitive change, where that change is measurable by changes to brain tissue actions, either neurophysiological or through muscle actions caused by the brain and the muscles resulting effects on the environment, for example, speech sounds.

Currently available helmets use accelerometers to measure the forces that the helmet, and therefore the head of the user, experiences. These accelerometers can be used to indicate when a force experienced by a helmet may be sufficiently large so as to pose a risk of a concussion to the user. However, currently available helmets are prone to providing false positives which can lead to unnecessary downtime for the user of the helmet. In addition, currently available helmets do not include any methods for tracking and analyzing impact data, other than indicating the occurrence of a potentially severe impact.

### SUMMARY

In accordance with an embodiment, a method for monitoring impacts between users of uniforms for concussion analysis includes monitoring one or more sensors in a uniform of a user, determining whether the user experienced an impact and storing data from the one or more sensors associated with the impact in a memory. Aspects also include transmitting a user identification code associated with the uniform and storing a second user identification code that is associated with another uniform involved in the impact in the memory.

In accordance with another embodiment, a system for monitoring impacts between users of uniforms for concussion analysis includes one or more sensors and a processor. The processor is configured to perform a method that includes monitoring one or more sensors in a uniform of a user, determining whether the user experienced an impact and storing data from the one or more sensors associated with the impact in a memory. Aspects also include transmitting a user identification code associated with the uniform and storing a second user identification code that is associated with another uniform involved in the impact in the memory.

In accordance with a further embodiment, a computer program product for monitoring impacts between users of uniforms for concussion analysis includes a non-transitory storage medium readable by a processing circuit and storing instructions for execution by the processing circuit for performing a method. The method includes monitoring one or more sensors in a uniform of a user, determining whether the user experienced an impact and storing data from the one or more sensors associated with the impact in a memory. Aspects also include transmitting a user identification code associated with the uniform and storing a second user identification code that is associated with another uniform involved in the impact in the memory.

### BRIEF DESCRIPTION OF THE DRAWINGS

The subject matter which is regarded as the invention is particularly pointed out and distinctly claimed in the claims at the conclusion of the specification. The forgoing and other features, and advantages of the invention are apparent from the following detailed description taken in conjunction with the accompanying drawings in which:

FIG. 1 is a block diagram illustrating one example of a processing system for practice of the teachings herein;

FIG. 2 is a block diagram illustrating a uniform in accordance with an exemplary embodiment;

FIG. 3 is a flow diagram of a method for monitoring impacts between users of uniforms for concussion analysis in accordance with an exemplary embodiment;

FIG. 4 is a block diagram illustrating a system for monitoring impacts between users of uniforms for concussion analysis in accordance with an exemplary embodiment; and

FIG. 5 is a block diagram illustrating an impact database in accordance with an exemplary embodiment.

### DETAILED DESCRIPTION

In accordance with exemplary embodiments of the disclosure, methods, systems and computer program products for using sensors in a helmet, or another suitable piece of a uniform, to monitor impacts between players for concussion analysis are provided. In exemplary embodiments, the sensors may include one or more of accelerometers, gyroscopes, or the like. In general, the outputs of the sensors are used to monitor one or more physical characteristics or actions of the user for signs of an impact involving the user. Once an impact is detected, the uniform records the data from the sensors associated with the impact and transmits a user identification code. In addition, after an impact is detected the uniform records a user identification transmitted by the other player involved in the impact. In exemplary embodiments, the uniform may be configured to store an impact database of all impacts experienced by the uniform or it may be configured to transmit the data associated with the impacts experienced by the uniform to a separate processing system, which may responsively update an impact database. In exemplary embodiments, the impact database may be analyzed to identify concussion risks associated with individual players, teams, positions and the like. The analysis of the impact database may include the creation and analysis of a hit graph that graphically illustrates the data in the impact database for visual analysis.

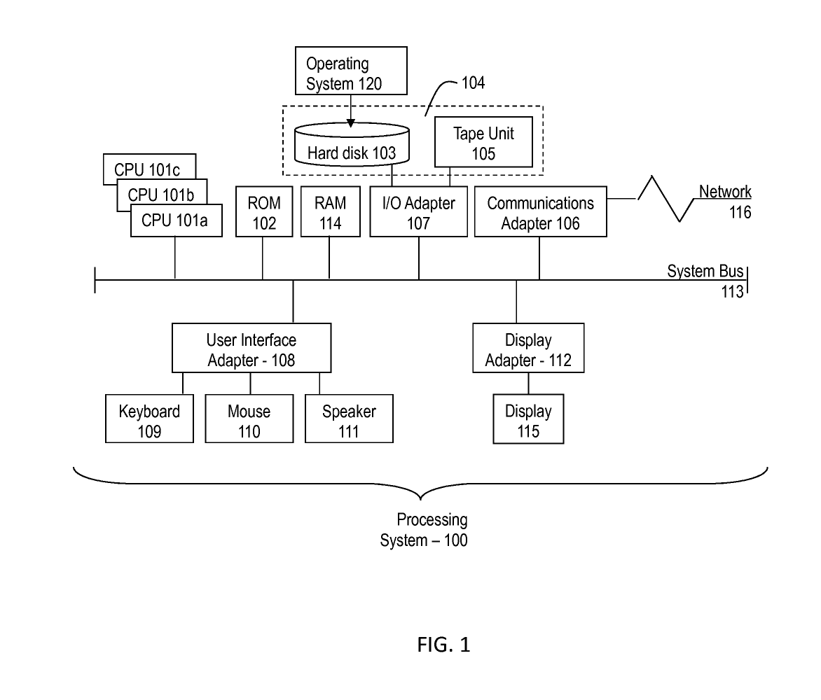

Referring now to FIG. 1, there is shown an embodiment of a processing system 100 for implementing the teachings herein. In this embodiment, the system 100 has one or more central processing units (processors) 101a, 101b, 101c, etc. (collectively or generically referred to as processor(s) 101). In one embodiment, each processor 101 may include a reduced instruction set computer (RISC) microprocessor. Processors 101 are coupled to system memory 114 and various other components via a system bus 113. Read only memory (ROM) 102 is coupled to the system bus 113 and may include a basic input/output system (BIOS), which controls certain basic functions of system 100.

FIG. 1 further depicts an input/output (I/O) adapter 107 and a network adapter 106 coupled to the system bus 113. I/O adapter 107 may be a small computer system interface (SCSI) adapter that communicates with a hard disk 103 and/or tape storage drive 105 or any other similar component. I/O adapter 107, hard disk 103, and tape storage device 105 are collectively referred to herein as mass storage 104. Operating system 120 for execution on the processing system 100 may be stored in mass storage 104. A network adapter 106 interconnects bus 113 with an outside network 116 enabling data processing system 100 to communicate with other such systems. A screen (e.g., a display monitor) 115 is connected to system bus 113 by display adaptor 112, which may include a graphics adapter to improve the performance of graphics intensive applications and a video controller. In one embodiment, adapters 107, 106, and 112 may be connected to one or more I/O busses that are connected to system bus 113 via an intermediate bus bridge (not shown). Suitable I/O buses for connecting peripheral devices such as hard disk controllers, network adapters, and graphics adapters typically include common protocols, such as the Peripheral Component Interconnect (PCI). Additional input/output devices are shown as connected to system bus 113 via user interface adapter 108 and display adapter 112. A keyboard 109, mouse 110, and speaker 111 all interconnected to bus 113 via user interface adapter 108, which may include, for example, a Super I/O chip integrating multiple device adapters into a single integrated circuit.

Thus, as configured in FIG. 1, the system 100 includes processing capability in the form of processors 101, storage capability including system memory 114 and mass storage 104, input means such as keyboard 109 and mouse 110, and output capability including speaker 111 and display 115. In one embodiment, a portion of system memory 114 and mass storage 104 collectively store an operating system such as the AIX® operating system from IBM Corporation to coordinate the functions of the various components shown in FIG. 1.

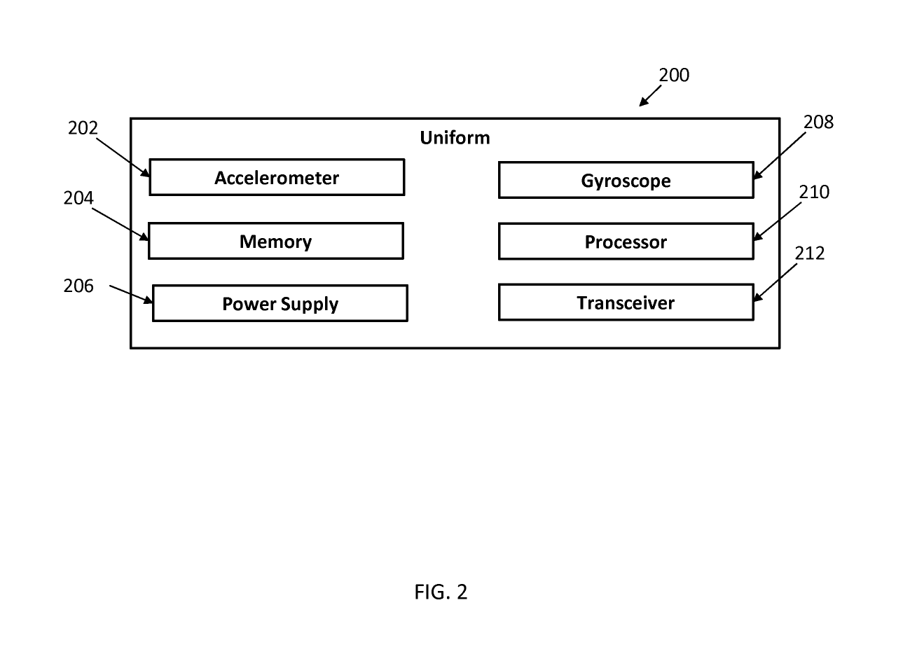

Referring now to FIG. 2, a block diagram illustrating a uniform 200 in accordance with an exemplary embodiment is shown. As used herein, a “uniform” is an outfit worn by individual while participating in an activity. The term uniform may include, but is not intended to be limited to, a helmet. In exemplary embodiments, the uniform 200 includes one or more of the following: an accelerometer 202, a memory 204, a power supply 206, a gyroscope 208, a processor 210, and a transceiver 212. In exemplary embodiments, the power supply 206 may be a battery configured to provide power to one or more of the accelerometer 202, the memory 204, the gyroscope 208, the processor 210 and the transceiver 212.

The processor 210 is configured to receive an output from one or more of the accelerometer 202 and the gyroscope 208 and to determine if a user of the uniform may have experienced an impact based on the inputs received. Upon making a determination that the user of the uniform has experienced an impact, the processor 210 records the data received from the sensors associated with the impact in the memory 204. In exemplary embodiments, upon detecting an impact, the uniform 200 utilizes the transceiver to transmit a user identification code associated with the uniform 200. In addition, the transceiver is configured to receive a user identification code from other uniforms involved in the impact. In exemplary embodiments, the uniform 200 stores the received user identification code in the memory 204 along with the corresponding data received from the sensors associated with the impact. The processor 210 may be configured to perform statistical and or graphical analysis on the stored impact data.

In one embodiment, the transceiver 212 includes a short range wireless transmitter that is configured to broadcast the user identification code associated with the uniform 200 to the immediate vicinity of the uniform 200 so that the uniform of another individual involved in the impact may receive the user identification code and so that the likelihood of reception of the user identification code by other individual not involved in the impact is minimized. For example, the transceiver 212 may broadcast the user identification code such that it can only be received by other uniforms within one to two feet of the uniform 200.

In another embodiment, the uniform 200 may be configured to exchange user identification codes with another uniform only when the two uniforms, or users, are in actual physical contact. In these embodiments, the transceiver 212 may include electrostatic materials that are configured to transmit and receive user identification codes with another uniform when the two uniforms are in physical contact. In one example, a Personal Area Networks (PANs) can be used to exchange user identification codes between uniforms disposed on and near the human body. For example, two individuals wearing uniforms make contact an electric circuit is completed, allowing picoamp signals to pass from the transceiver 212 of the uniform through the body of a first user to the body of the second user.

In exemplary embodiments, these PANs can exchange digital information by capacitively coupling picoamp currents through the bodies of the users. In addition, to exchanging identification codes and the uniform 200 may also be configured to exchange data collected for all of the impacts experienced by the uniform. For example, the uniform 200 can exchange a hit graph contain data for all impacts experienced by the uniform 200 anytime one uniform 200 makes contact with another uniform. Accordingly, the impact data available to each of the uniforms will spread as the uniforms make contact with one another.

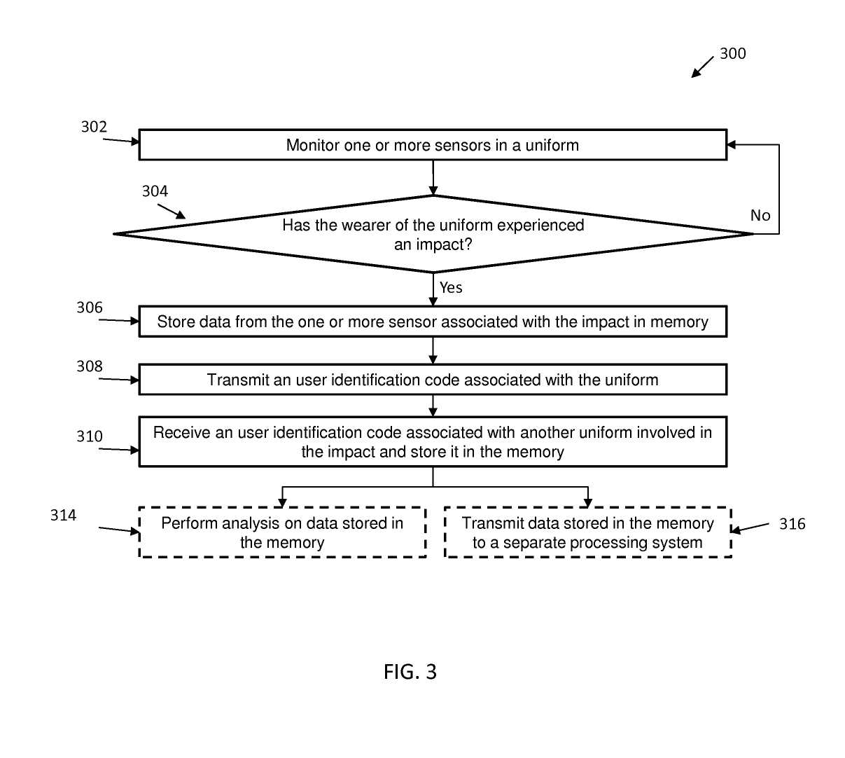

Referring now to FIG. 3, a flow diagram of a method 300 for monitoring impacts between users of uniforms for concussion analysis in accordance with an exemplary embodiment is shown. As shown at block 302, the method 300 includes monitoring one or more sensors in a uniform. In exemplary embodiments, the one or more sensors may include an accelerometer and/or a gyroscope. Next, as shown at decision block 304, the method 300 includes determining whether wearer of the uniform experienced an impact based on the output of the one or more sensors. In exemplary embodiments, the determination of whether wearer of the uniform experienced an impact may include comparing the output from the one or more sensors to threshold levels, which may be selected based on user specific data. If the wearer of the uniform has not experienced an impact, the method returns to block 302 and continues to monitor one or more sensors in a uniform. If the wearer of the uniform has experienced an impact, the method proceeds to block 306 and includes storing the data from the one or more sensor associated with the impact in the memory. In exemplary embodiments, the memory of the uniform may store an impact database that is used to store all available data regarding impacts experienced by the wearer of the uniform.

Continuing with reference to FIG. 3, as shown at block 308, the method 300 includes transmitting a user identification code associated with the uniform. In exemplary embodiments, the user identification code may be wireless transmitted or it may be transmitted through physical contact between two uniforms. Next, as shown at block 310, the method 300 includes receiving a user identification code associated with another uniform involved in the impact and stores it in the memory. In exemplary embodiments, the user identification code may be wireless received or it may be received through physical contact between two uniforms. Optionally, the method 300 may include performing analysis on the impact data stored in the memory, as shown at block 312. Likewise, the method 300 may include transmitting data stored in the memory to a separate processing system, as shown at block 314.

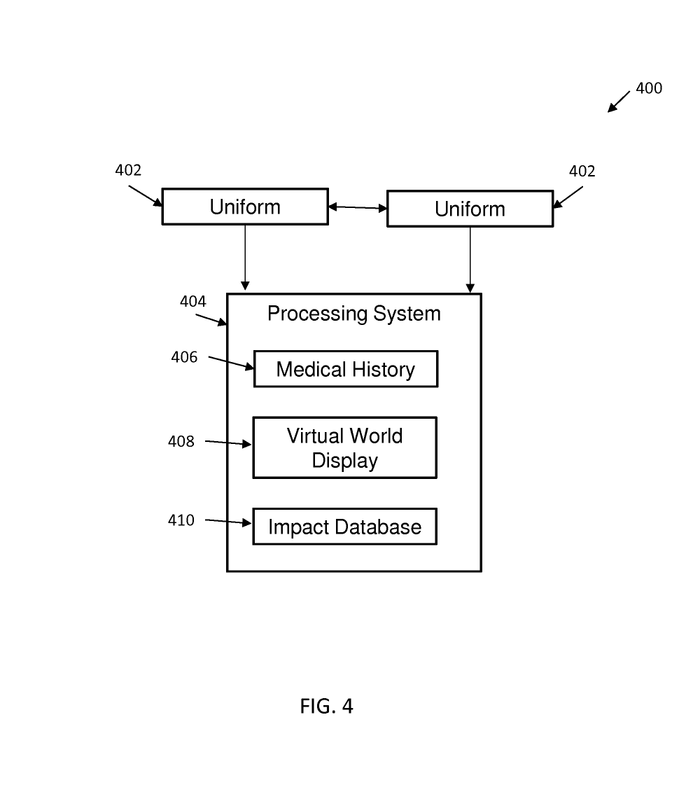

Referring now to FIG. 4, a block diagram illustrating a system 400 for monitoring impacts between users of uniforms for concussion analysis in accordance with an exemplary embodiment is shown. As illustrated, the system 400 includes one or more uniforms 402, such as the one shown and described above with reference to FIG. 2, and a processing system 404, such as the one shown and described above with reference to FIG. 1. The processing system 404 is configured to communicate with the uniforms 402 and is also configured to store the medical history 406 of the users of the uniforms 402. In exemplary embodiments, the medical history 406 of the users of uniforms 402 may be used by the uniform 402 in setting the threshold levels for determining whether an impact has occurred and/or during the analysis of impact data stored in the memory of the uniform.

In addition, the processing system 404 may include a virtual world display 408 that is configured to provide a display a real-time status of each of the users of the uniforms. In exemplary embodiments, the status may include, the category of play of each user, any indications that the user may have suffered a traumatic brain injury, a duration of play of the user, a duration that the user has been in the current category of play, a summary or sampling of the impact data for the user, or the like.

In exemplary embodiments, the user's history of collision or medical concerns may be used to determine a traumatic brain injury risk assessment, either by the embedded processor or the separate processing system. In addition, the uniform may be configured to provide a real-time feed of the user's cognitive state to increase the confidence level of the need for a particular alert or indication. In exemplary embodiments, an aggregate indication may be used to summarize an overall state of a group of players. This may also help to potentially identify area of risk in the dynamics of player-player interaction, overly aggressive players, playing field conditions, etc. In exemplary embodiments, an automatic feed from a user's history of collision or medical concerns may also be provided to a processor of the helmet in order to update an impact risk model for each category of play. In addition, the processing system 404 may receive a real-time feed of the user's cognitive state, which can be used to update the risk models used by the helmets. The risk models may also be sent to the virtual world display 408 of the game and players, which allows the sports staff health professionals to visualize the nature of potential problems. In exemplary embodiments, the processing system 404 may store an impact database 410 that includes impact data received from the one or more uniforms 402.

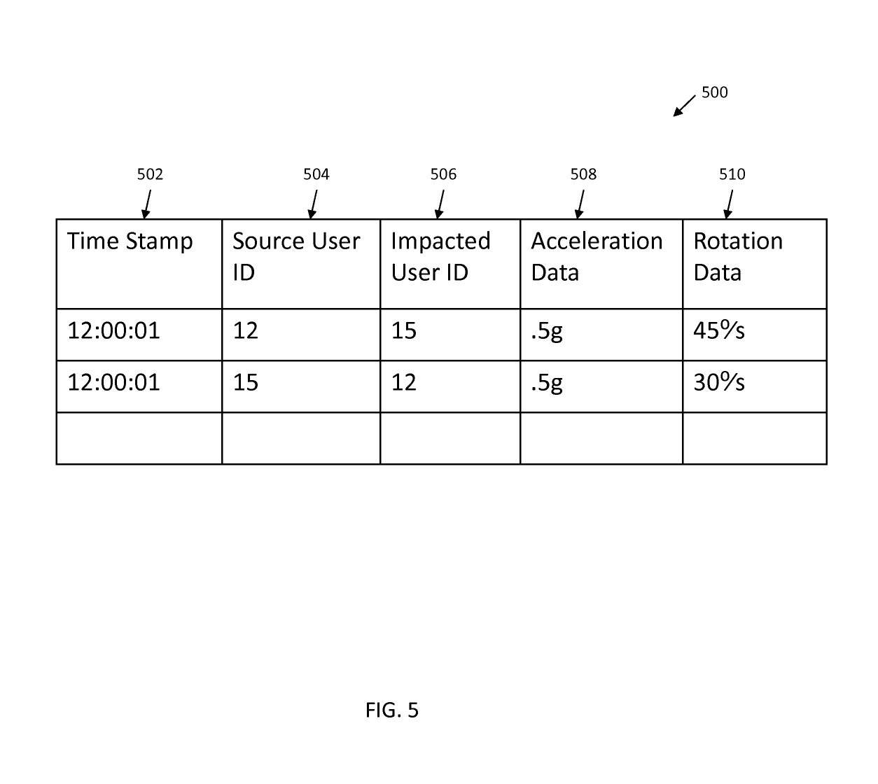

Referring now to FIG. 5, a block diagram illustrating an impact database 500 in accordance with an exemplary embodiment is shown. As illustrated, the impact database 500 includes a plurality of entries that each corresponds to a hit experienced by one of the uniforms. Each entry includes a timestamp 502, a source user identification code 504, an impacted user identification code 506, acceleration data 508 and rotation data 510.

In exemplary embodiments, the impact database can be analyzed by constructing hit graphs for each uniform and analyzing the hit graphs to identify any increased concussion risk for each user. For example, the hit graph for a specific user may indicate that the user is experiencing an abnormally high amount of impacts or impacts of above average intensity. In addition, the impact database can be analyzed by constructing a combined hit graph the uniforms and analyzing the hit graph to identify any increased concussion risk for one or more users. In exemplary embodiments, the topology of the hit graphs can be used to predict concussive risk during play and an ameliorative action can deployed, such as a warning, change in helmet parameters, or the like.

The present invention may be a system, a method, and/or a computer program product. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

What is claimed is:

1. A method for monitoring impacts between users of uniforms for concussion analysis, the method comprising:
monitoring one or more sensors in a uniform of a user;
determining whether the user experienced an impact;
storing data from the one or more sensors associated with the impact in a memory;
transmitting a user identification code associated with the uniform; and
storing a second user identification code that is associated with another uniform involved in the impact in the memory.

2. The method of claim 1, further comprising performing analysis on data stored in the memory.

3. The method of claim 1, wherein transmitting the user identification code associated with the uniform includes transmitting the user identification code through a personal area network created by contact between the users of uniforms.

4. The method of claim 1, further comprising transmitting, by the uniform, data stored in the memory to a separate processing system.

5. The method of claim 4, further comprising creating an impact database by the separate processing system from the data received from multiple uniforms.

6. The method of claim 5, wherein the impact database includes entries for all detected impacts between users of uniforms and wherein the method further comprises performing analysis on the impact database to identify a concussion risk for each user.

7. The method of claim 1, wherein determining whether the user experienced the impact includes comparing an output of the one or more sensors to one or more threshold levels associated with a medical history of the user.
